(19) United States
(12) Patent Application Publication
(10) Pub. No.: US 2007/0066893 A1
(43) Pub. Date: Mar. 22, 2007

(54) Method

(76) Inventors: Morten Eriksen, Oslo (NO); Lars Hoff, Tolvsrod (NO); Henrik Rasmussen, Oslo (NO); Audun Tornes, Oslo (NO); Jonny Ostensen, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/558,597

(22) PCT Filed: Dec. 3, 2004
(86) PCT No.: PCT/NO04/00372

§ 371 Date: Nov. 29, 2005

(30) Foreign Application Priority Data
Dec. 4, 2003 (NO) 20035401

(51) Int. Cl. A61B 6/00

(52) U.S. Cl. 600/436

(57) ABSTRACT

The present invention relates to a method of processing ultrasound images from a subject preadministered with an ultrasound contrast agent wherein quantitative measures of the contrast enhancement pattern of said lymph nodes are generated. The lymphatic system is made of vessels or ducts that begin in tissues and are designed to carry lymph fluid to local lymph nodes where the fluid is filtered and processed and sent to the next lymph node down the line until the fluid reaches the thoracic duct where it enters the blood stream. Lymph fluid which enters the lymph vessels carries with it substances and materials from the tissue, e.g. antigens, particles and cells. The lymph nodes process the lymph fluid by sieving it and macrophages inside the nodes remove particulate and cell material carried by the lymph fluid via phagocytosis.

### METHOD

The present invention relates to a method of processing ultrasound images from a subject preadministered with an ultrasound contrast agent wherein quantitative measures of the contrast enhancement pattern of said lymph nodes are generated.

The lymphatic system is made of vessels or ducts that begin in tissues and are designed to carry lymph fluid to local lymph nodes where the fluid is filtered and processed and sent to the next lymph node down the line until the fluid reaches the thoracic duct where it enters the blood stream. Lymph fluid which enters the lymph vessels carries with it substances and materials from the tissue, e.g. antigens, particles and cells. The lymph nodes process the lymph fluid by sieving it and macrophages inside the nodes remove particulate and cell material carried by the lymph fluid via phagocytosis.

When cancer occurs in tissues or organs, its loose matrix may allow the dislodging of cells that gain access to the lymphatic system, become trapped in the lymph node and grow. In early stages of cancer development in the node, the cancer remains limited to the node. However, in time, the nodal deposit can grow to totally replace the node and/or can spread downstream to the next node. The lymph nodes that drain the tissue or organ of interest (i.e., the cancerous tissue) are called the regional lymph nodes and the first node that traps the cancer is called the sentinel lymph node.

In the past, it has been normal practice in some situations to remove all lymph nodes potentially harbouring neoplastic cells metastasised from a tumour. A high morbidity rate is associated with this practice. Thus, several methods were developed to identify and biopsy lymph nodes, especially the sentinel lymph nodes. If the sentinel lymph node is free of neoplastic cells, then further lymph node biopsies and lymph node dissections can be avoided. Generally, sentinel and other lymph nodes have been identified by injecting a marking agent into the tumour-bearing tissue and tracing the pathway of the marking system through the lymphatic system.

Possible marking agents are for instance visible marking agents such as dyes (A. E. Giuliano et al., Ann. Surg. 220, 1994, 391-401) or radiopharmaceutical compounds as disclosed in U.S. Pat. No. 5,732,704. Further, contrast agents have been used for lymph node imaging and/or sentinel lymph node detection (see for instance WO-A-00/45855 and WO-A-00/38579).

However, in order to characterise the identified lymph nodes, i.e. to -discriminate between normal and malignant lymph nodes, the nodes are usually at least partially removed and histologically assessed for the presence or absence of malignant cells. Because of its invasive nature such a procedure means additional physiological stress for the patient.

Mattrey et al. noticed during sentinel lymph node ultrasound imaging that cancer within the node did not fill with contrast agent material, thus leaving a filling defect. However, it was stated that further work is required for confirmation. (R. Mattrey et al., Academic Radiology 9, 2002, S231-S235).

International patent application no. PCT/NO03/00328 discloses a method for identifying a sentinel lymph node using microbubbles as contrast agents and ultrasound detection. It is further described that benign and malignant sentinel lymph nodes may be discriminated due to their different enhancement patterns: while benign sentinel lymph nodes appear uniformly echogenic, malignant sentinel lymph nodes demonstrate a heterogenic enhancement pattern with both areas of increased echogenity and areas that do not enhance.

A disadvantage of a method of discriminating between normal and malignant lymph nodes based on the observations described in the two paragraphs above is that the discrimination is based on visual assessment only and hence requires a physician who is especially trained to do this visual assessment.

Accordingly, there is a need for a non-invasive, simple and more reliable method to discriminate between normal and malignant lymph nodes that at the same time is safe for the patient.

It has surprisingly been found that the processing of ultrasound images of lymph nodes from a subject preadministered with an ultrasound contrast agent wherein quantitative measures of the contrast enhancement pattern of said lymph nodes are generated can be used to discriminate between normal and malignant lymph nodes. By providing a quantitative measure, the discrimination is based on objective criteria rather than on subjective visual assessment alone. Further, the analysis of the generated data (i.e. the quantitative measures) may be carried out using automated tools like software programs which further eliminates false positive or negative results associated with visual assessment only. The method is applicable to all areas of the body where sonolymphography is possible; areas of particular interest are breast cancer, melanoma and neck tumours.

Hence the invention provides a method of processing ultrasound images of lymph nodes from a subject preadministered with an ultrasound contrast agent wherein quantitative measures of the contrast enhancement pattern of said lymph nodes are generated. This method is preferably used to discriminate between normal and malignant lymph nodes.

In the context of the present invention, “subject” means a vertebrate subject like a bird or a mammal and preferably a human being.

In the context of the present invention, the term “malignant” is interchangeably used with tumour tissue, cancerous, metastatic and the like. It denotes a condition which differs from the normal healthy condition.

In the context of the present invention, the term “normal” is interchangeably used with benign, healthy and the like. It denotes a non tumour/cancer/metastatic condition.

The ultrasound contrast agent the subject is preadministered with before the image processing according to the invention is performed may be any ultrasound contrast agent like free or stabilized gas, i.e. a gas, which for instance is surrounded by a matrix material and/or included into a microbubble, vesicle, liposome or a microcapsule. Further gas precursors might be used as ultrasound agents for preadministration. The term “gas precursor” denotes a material that is a liquid or a solid at ambient temperature and pressure and changes phase from liquid to gas at the relevant temperature, e.g. the body temperature of the subject. When referring to “gas” and “gas precursor”, it will be understood that mixtures of gases and gas precursors fall within the definition. In a preferred embodiment, the subject is preadministered with a particulate ultrasound contrast agent, preferably with a particulate ultrasound contrast agent comprising a matrix material and a gas, a gas precursor or mixtures thereof. In a further preferred embodiment, the particulate ultrasound contrast agent comprises microbubbles, vesicles, liposomes or microcapsules preferably comprising a gas, a gas precursor or mixtures thereof.

Such preferred particulate ultrasound contrast agents comprise one or more matrix materials from the group consisting of carbohydrates, proteins, lipids, natural or synthetic polymeric materials, saccharides, peptides, proteinaceous materials and the like or combinations thereof.

In a particularly preferred embodiment, microbubbles comprising a shell and a gas or gas precursor and having a mean particle size of about 0.25-15 μm in diameter and a pressure stability of at least 50% at a pressure of 120 mm Hg are used in connection with the method according to the invention, i.e. for the preadministration of the subject. Such microbubbles are especially useful when the method according to the invention is used for the discrimination between normal and malignant sentinel lymph nodes as these microbubbles remain intact upon injection. They are not only easily taken up by the lymphatic system and the sentinel lymph node but they are also retained in the sentinel lymph node and remain stable, thus allowing sensitive and effective ultrasound detection.

The term “shell” can be interchangeably used with the term “wall” or “membrane” and means material surrounding or defining a microbubble. The shell may be in the form of one or more layers, preferably in the form of a single monolayer or a bilayer (unilamellar), and the mono- or bilayer may be used to form one or more mono- or bilayers (oligo- or multilamellar). In the case of more than one mono- or bilayer, the mono- or bilayers are preferably concentric. Suitably, the shell is formulated from lipids, natural or synthetic polymeric materials, proteinaceous materials, carbohydrates, saccharides, and the like or combinations thereof. In a preferred embodiment, the shell has an overall negative or positive net charge. Thus, the shell may be composed of or comprise polymeric material or proteinaceous material having an excess of negative or positive charges or the shell may be composed of or comprise negatively or positively charged lipids. Alternatively, the shell may be composed of or comprise neutral polymeric, proteinaceous or lipid materials combined with incorporation or surface modification using negatively or positively charged components that give an overall net charge. Preferably, the shell is composed of or comprises lipids, more preferably phospholipids, e.g. phosphatidylcholines, preferably dilauroyl phosphatidylcholine, dimyristoyl phosphatidylcholine, diheptadecanoyl phosphatidylcholine, dipalmitoyl phospatidylcholine, distearoyl phosphatidylcholine, diarachidoyl phosphatidylcholine or dibehenoyl phosphatidylcholine, phosphatidylserines, preferably dipalmitoyl or distearoyl phosphatidylserine, phosphatidylglycerols, preferably dipalmitoyl or distearoyl phosphatidylglycerol, phosphatidylethanolamines, preferably dipalmitoyl or distearoyl phosphatidylethanolamine, phosphatidylinositols, preferably dipalmitoyl or distearoyl phosphatidylinositol, phosphatidic acid, preferably dipalmitoyl or distearoyl phosphatidic acid, cardiolipins or any mixture of the foregoing named compounds, optionally in a mixture with cholesterol, cholesterol sulfate, cholesteryl hemisuccinate, N-palmitoyl homocystein, palmitic acid, oleic acid, stearic acid or arachidic acid. Alternatively, the shell may also be composed of or comprise fluorinated analogues of the above-mentioned lipids. In another preferred embodiment, the shell is composed of or comprises the above-mentioned lipids which are covalently linked to hydrophilic polymers such as polyethylene glycol (PEG), preferably PEG 2000-8000, e.g. dipalmitoyl or distearoyl phosphatidylethanolamine-polyethyleneglycol 5000. In a particularly preferred embodiment, the shell is composed of or comprises—preferably in an amount of from 50 to 100%, more preferably in an amount of 70 to 90%—negatively charged phospholipids, more preferably negatively charged phospholipids based on fatty acids having at least 14 carbon atoms, e.g. myristic acid, palmitic acid, stearic acid oleic acid or arachidic acid, e.g. dipalmitoyl phosphatidylglycerol or dipalmitoyl phosphatidylethanolamine-PEG. In another preferred embodiment, the shell comprises—preferably in an amount of from 1 to 20%—positively charged synthetic lipids, e.g. cationic lipids normally used in nucleic acid delivery such as DOTAP (N-1(-(2,3-dioleoyloxy)propyl)-N,N,N-trimethylammoniumethyl sulphate), DOTMA (N-(1-(2,3-dioleoyloxy)-propyl)-N,N,N-trimethylammonium chloride), DOGS (dioctadecylamidoglycyl spermine) and the like. Alternatively, the shell is composed of or comprises lipopeptides, for examples lipopeptides as described in WO-A-99/55383, the content of which is incorporated herein by reference.

Suitably, the microbubbles preferred for use for preadministration comprise air, nitrogen and fluorinated compounds, either partially fluorinated or fully fluorinated (perfluorinated compounds), as pure compounds or mixtures thereof. In a preferred embodiment, the microbubbles comprise perfluorinated compounds, e.g. perfluoropropane, perfluorobutane, perfluoropentane, perfluorohexane, sulfur hexafluoride and the like, optionally in admixture with nitrogen.

The microbubbles preferred for use for preadministration have a mean particle size of 0.25-15 μm in diameter, preferably 0.5-7 μm, particularly preferably 1-5 μm. They have a pressure stability of at least 50% at a pressure of 120 mm Hg, preferably at least 70%, more preferred at least 85% and most preferred of at least 95%. The pressure required for administration of such microbubbles into (soft) tissue might be substantial thus the ultrasound contrast agent must be able to resist such pressures without being destroyed. Hence a pressure stability of at least 50% at a pressure of 120 mm Hg is a preferred property of such microbubbles.

In the context of the invention, the term “pressure stability of at least 50%” means that the acoustic attenuation efficacy of the microbubbles after being exposed to a pressure of 120 mm Hg is at least 50% of the acoustic attenuation efficacy of said microbubbles before being exposed to said pressure. Hence, by comparing the acoustic attenuation efficacy of the microbubbles before and after exposure to pressure, a measure of the microbubble's ultrasound imaging efficacy can be obtained. The acoustic attenuation efficacy can be determined by measuring the dampening (dB/cm) of a sound beam going through a diluted suspension of the microbubble sample using one or two broad band transducers with centre frequencies 3.5 and/or 5.0 MHz. Transmission is measured by pulse-echo technique; short pulses of sound are emitted from the transducer and traversed through a measuring cell compartment before being reflected from the back wall of the compartment and received again by the emitting transducer. The pulses are digitised by an oscilloscope and frequency spectra are calculated by Fourier transformation. To compensate for transmission path and transducer characteristics, the spectra are normalised to spectra of the pure diluent. A detailed description of the measurement of attenuation spectra and a suitable system setup for is described in L. Hoff, Acoustic Characterization of Contrast Agents for Medical Ultrasound Imaging, Kluwer Academic Publishers, 2001, chapter 4, page 99-109, the disclosure of which is incorporated herein by reference. The analysis is normally conducted in the range of 0° C. to 50° C., preferably at ambient room temperature. In a first step of the analysis, a reference spectrum is taken from the diluent. Suitable diluents are free of air bubbles and any liquids in which the microbubbles are stable could be used, preferable diluents are isotonic saline solution like Isoton II (Coulter Electronics Ltd. Luton, UK), a 0.9% saline solution comprising a phosphate buffer and a detergent to reduce surface tension. In a next step, the microbubble sample is mixed with the diluent and one or more attenuation spectra are measured at ambient pressure. The concentration of the microbubble sample is adapted to the size of the microbubbles. Preferably, the dilution factor is such that the attenuation from the microbubble sample is between 15 and 20 dB, i.e. about 3 dB/cm. This typically means that the microbubbles are diluted by a factor 10³ to 10⁴. In a next step, the pressure is raised to 120 mm Hg and one or more attenuation spectra are measured. In order to determine the pressure stability, the acoustic attenuation efficacy of the microbubble sample before pressure is set to 100% thus allowing the calculation of the relative acoustic attenuation efficacy of the microbubble sample after pressure.

In a further preferred embodiment, the microbubbles preferred for use for preadministration are stable for pressure variations associated with ultrasound imaging of a mechanical index of at least 0.2. A method to determine the microbubble stability associated with ultrasound imaging pressures is described in W. T. Shi et al., Ultrasound in Med. & Biol., Vol. 26, 2000, 1-11.

Suitably, the microbubbles preferred for use for preadministration are echogenic, i.e. they are capable of scattering or reflecting ultrasound waves. Preferably, the microbubbles are adapted to return a signal at a frequency different from the transmit frequency of the ultrasound pulse.

The microbubbles preferred for use for preadministration can be prepared in a variety of ways which are readily apparent to those skilled in the art, including, e.g. shaking, vortexing, sonication, extrusion, repeated freezing and thawing cycles, extrusion under pressure through pores of defined size and spray drying. For example for lipid comprising microbubbles, the lipid-containing medium may be subjected to any appropriate emulsion generating technique, e.g. sonication, high pressure homogenisation, high shear mixing, in the presence of the selected gas or gas precursor. The gas employed in the emulsification step need not to be the same as in the final microbubble. Thus, most of this gas may be removed during a subsequent lyophilisation step and residual gas may be removed by evacuation of the dried product, to which an atmosphere or overpressure of the desired end product gas may then be applied (see for example WO-A-97/29783).

Other methods of forming such microbubbles include the formation of protein comprising microbubbles (EP-A-359 246 and US 4,718,433), the formation of lipid containing microbubbles (U.S. Pat. No. 4,684,479) and the formation of liposomal microbubbles (U.S. Pat. No. 5,088,499; U.S. Pat. No. 5,123,414 and WO-A-94/28874).

The ultrasound contrast agent is preferably preadministered in form of a preparation, preferably in form of a liquid preparation. In the following, the terms “ultrasound contrast agent” and “ultrasound contrast agent preparation” are used interchangeably.

Preparations useful for preadministration comprise an ultrasound contrast agent, preferably a particulate ultrasound contrast agent, more preferably microbubbles and one or more components selected form the group consisting of osmotic agents, stabilisers, surfactants, buffers, viscosity modulators, emulsifiers, solubilising agents, suspending agents, wetting agents, antioxidants, viscosity increasing agents, tonicity raising agents, salts, sugars and the like. Such components are added to ensure maximum life and effectiveness of the ultrasound contrast agent. Additionally, considerations as sterility, isotonicity and biocompatibility may govern the use of such components.

Suitable viscosity modulators include, for example, carbohydrates and their phosphorylated and sulfonated derivatives; polyethers, preferably with molecular weight ranges between 400 and 100.000 and di- and trihydroxy alkanes and their polymers, preferably with molecular weight ranges between 200 and 50.000.

Suitable emulsifying and/or solubilizing agents include, for example, acacia, cholesterol, glyceryl monostearate, lanolin alcohols, lecithin, mono-and diglycerides, ethanolamine, diethanolamine oleic acid, oleyl alcohol, poloxamer, for example, poloxamer 188, poloxamer 184, and poloxamer 181, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, emulsifying wax, and the like.

Suitable suspending and/or viscosity-increasing agents include, for example, acacia, agar, alginic acid, aluminum mono-stearate, bentonite, magma, carbomer 934 P, cellulose, methylcellulose, carboxymethylcellulose, hydroxyethyl cellulose, hydroxypropyl methylcellulose, carrageenan, dextran, gelatin, guar gum, locust bean gum, veegum, magnesium-aluminum-silicate, silicon dioxide, zeolites, pectin, polyethylene oxide, povidone, propylene glycol alginate, sodium alginate, tragacanth, xanthan gum, alpha-d-gluconolactone, glycerol and mannitol.

Suitable suspending agents are for example polyethylene glycol (PEG), polyvinylpyrrolidone (PVP), polyvinyl alcohol (PVA), polypropylene glycol (PPG), polysorbate and the like.

Suitable tonicity raising agents which stabilise and add tonicity are, for example, sorbitol, mannitol, trehalose, sucrose, propylene glycol and glycerol.

The preparations comprising the ultrasound contrast agent for use for preadministration may further comprise dyes or biologically active agents, preferably selected from the group consisting of analgesics, antibiotics, leukotriene inhibitors or antagonists, antihistamines, anttiinflammatories, antineoplastics, anticholinergics, anesthetics, enzymes, steroids, genetic material, viral vectors, antisense agents, proteins and peptides.

In a preferred embodiment, the preparations further comprise compounds which promote macrophage uptake, e.g. mannans, for example zymosan, mannose-containing oligo- and polysaccharides, Fc (fragment crystallizable) portions of immunoglobulin molecules, complement components, for example C3b or C3bi, ligands for scavenger receptors, ligands for toll-like receptors, ligands for LRPs (LDL-receptor-like proteins), bacterial glycopeptides or lipopolysaccharides for example bleomycin or endotoxin.

The preparations are preferably sterile injectable formulations like suspensions or emulsions comprising suitable carriers including non-toxic parenterally-acceptable aqueous or non aqueous solutions and the components and/or compounds described above. Preferred carriers are water or saline.

The preparations for use for preadministration may be formulated according to known methods. In a preferred embodiment, the preparations are manufactured immediately before use, i.e. before administration to the subject. Thus, a dry ultrasound contrast agent, preferably a microbubble product, may be mixed with a suitable carrier and one or more of the above mentioned compounds and components. In another preferred embodiment, microbubble preparations are manufactured immediately before use and the microbubbles are generated in situ during the manufacture of the preparation, e.g. by adding a suitable carrier to a vial that contains the desired gas and the components comprising the shell of the microbubbles and agitating this mixture. The preparations for use for preadministration are preferably sterilised before administration to the subject and/or are manufactured from sterile starting materials. Sterilisation of the preparations may be achieved by filtration through a bacteria-retaining filter, by incorporating sterilising agents, by irridation and the like.

Preadministration of the ultrasound contrast agent before image acquisition can be done in various fashions which are not intravascular. Parenteral preadministration is preferred and includes but is not limited to the following routes: intramuscular, percutaneous, directly in a lymphatic vessel, interstitially, intraperitoneal, intrathecal, subcutaneous, intrasynovial, transepithelial (including transdermal) dermal, intradermal, subdermal, in a tumour or pathologic process itself, and the like. Preferably, the ultrasound contrast agent is interstitially preadministered, preferably by interstitial injection including subcutaneous and intradermal injection. In a preferred embodiment, the ultrasound contrast agent is injected in proximity to the tumour or (peritumoural). The ultrasound contrast agent can also be preadministered by combining injection of two or more parenteral modes, for example intramuscular, subcutaneous, and in the pathologic process, ensuring its accretion in the lymph node.

The ultrasound contrast agent will normally be preadministered at a site and by means that ensure that it is mobilised and taken up into the lymphatic circulation. This will vary with the system to be imaged. Multiple injection sites may be preferable in order to permit proper drainage to the regional lymph nodes under investigation. In some cases, injections around the circumference of a tumour or biopsy site are desired. In other cases, injection into a particular sheath or fossa is preferred. Injection into the webs of the fingers or toes is a common mode used to study peripheral lymphatics. The ultrasound contrast agent can be preadministered to the subject pre-operatively and/or intra-operatively to localise the lymph nodes. The method according to the invention allows immediate and real-time identification of the lymph nodes following preadministration of the ultrasound contrast agent in a region of interest as preadministration does not require significant lead time to reach the lymph nodes. Moreover, additional methodology can be employed to modify or alter the transport of the ultrasound contrast agent to the lymph nodes, including massaging the injection site or stimulating flow. Preferably, the site of injection of the ultrasound contrast agent will be massaged.

When the microbubbles preferred to use in the method of the invention are employed, the method may be used to locate the lymph node and to discriminate between normal and malignant sentinel nodes associated with breast tumour. Images of axillary, subclavian and supraclavicular nodes may be obtained by injecting the microbubble preparation into and around the tumour and below the skin adjacent to the tumour. A unilateral injection can be made in the subcostal site ipsilateral to the tumour, followed by bilateral lymph node imaging. By injecting the preparation in the vicinity of the tumour, the practitioner will know that the lymph duct involved and leading to the sentinel node will be directed toward the axillary, internal mammary, or supraclavicular chain wherein ultrasound detection is effected at appropriate times after each injection.

When using the method according to the invention to discriminate between normal and malignant sentinel nodes associated with breast tumour it is preferred to inject the microbubble preparation around the areola tissue of the breasts bilaterally, and then detecting the axillary, internal mammary, or supraclavicular chains. In addition to periareolar injection, interdigital administration of the preparation may be used for visualization of axillary lymphatics (see, DeLand et al., (1980), Cancer Res. 40:2997-3001). Combined interdigital and periareolar administration of the preparation can provide increased accuracy to demonstrate increased uptake in affected axillary nodes. Intratumoural injection of the microbubbles can also be performed in patients with breast cancer or melanoma.

The ultrasound contrast agent should be preadministered in an effective amount, i.e. in an amount which allows sufficient detection. Volumes of ultrasound contrast agent preparations in liquid form will normally vary being dependent upon, e.g., the site of preadministration, the concentration of the preparation, the number of injections, the composition of the preparation and/or the type of ultrasound contrast agent present therein and the properties specific for each individual subject.

After the preadministration, the ultrasound contrast agent is allowed to accumulate in the lymph nodes.

If preadministration is carried out in a way as described on pages 11 and 12 of the present application, ultrasound contrast agents generally do not require significant lead time to reach the lymph nodes and accumulate therein. This is especially true if microbubbles comprising a shell and a gas or gas precursor and having a mean particle size of about 0.25-15 μm in diameter and a pressure stability of at least 50% at a pressure of 120 mm Hg have been used as ultrasound contrast agents for preadministered. Such microbubbles are further described on page 4 of the application. Thus, immediate and real-time identification of the lymph nodes following administration of the ultrasound contrast agent is possible. Generally, the ultrasound contrast agent used for preadministration should be capable of accumulating in the lymph nodes in less than 60 minutes. In a preferred embodiment, the ultrasound contrast agent accumulates in the lymph nodes in less than 15 minutes and particularly preferably in less than 5 minutes.

With respect to the acquisition of ultrasound images, ultrasound imaging techniques contemplated for use in the present invention are well known in the art, and are described, for example, in McGahan and Goldberg, Diagnostic Ultrasound: A Logical Approach (Lippincott-Raven Publishers 1998), and in Frederick and Kremkau, Diagnostic Ultrasound: Principles and Instruments, (W B Saunders Co. 1998). Specific ultrasound imaging modes useful with the disclosed invention include harmonic or non-linear imaging, grey scale (B-mode), Doppler (including pulsed wave, power, flow, colour, amplitude, spectral and harmonic), 3-D imaging, gated imaging, and the like. With respect to non-linear imaging, it will be appreciated that the present invention is compatible with wideband harmonic imaging and pulse inversion harmonic imaging.

If one desires to use harmonic imaging and the ultrasound imaging machine is set to image at a particular frequency, the outgoing waveform supplied to the sonic transducer can be a numerical fraction of the imaging frequency (e.g., ½, ⅔, ⅓, and the like) or a whole number or fractional multiple of the imaging frequency (e.g., 2, 3, 4, and the like). With any particular combination of microbubble preparation and excitation frequency, certain harmonics will be dominant. The second harmonic is a common example. Those strongest harmonics are preferred for obvious reasons, although other harmonics or frequencies may be selected for reasons such as preparation of multiple images or elimination of background. Moreover several frequencies, including harmonic and non-harmonic frequencies or some combination thereof, may be simultaneously detected to provide the desired image. That is, in preferred embodiments any frequency other than the interrogation frequency may be used to provide the desired data. Of course, those skilled in the art will appreciate that dominant harmonics can be determined by simple empirical testing of the ultrasound contrast agent.

To detect the re-radiated ultrasound energy generated by the ultrasound contrast agent, a modified conventional ultrasound scanner system or commercially available non-linear imaging systems can be used. These systems are able to detect or select one or more or all of the new frequencies, or harmonics, radiated by the microbubble preparation for production of the ultrasound image. In other words, it detects a frequency different from the emitted frequency. Equipment suitable for harmonic ultrasound imaging is disclosed in WO-A-91/15999. Many conventional ultrasound imaging devices, however, utilise transducers capable of broad bandwidth operation, and the outgoing waveform sent to the transducer is software controlled. For this reason, reprogramming to emit a waveform different from the one detected is well within the level of skill in the art.

Although non-linear ultrasound imaging such as harmonic ultrasound imaging, second harmonic ultrasound imaging or preferably pulse inversion is particularly preferred for use in the disclosed method, other types of ultrasound conventional imaging such as B-mode (gray scale imaging) and F-mode (colour flow or Doppler imagingare also compatible and within the purview of the present invention.

In B-mode imaging, the ultrasound system typically transmits a series of beams, along scan lines, steered to scan a desired field of view. The ultrasound system typically steers “receive beams” in a manner that corresponds to the transmit beams. Data returned from each receive beam is communicated to an image display subsystem which reconstructs a two-dimensional gray scale image from the B-mode data and displays it on a console. Such series of pulses down a single line may be identical or may be of equal or unequal frequency or have a near 180 degree phase shift (inverted pulse) to promote the distinction of the ultrasound contrast agent from the surrounding tissues.

F-mode imaging is accomplished in a manner similar to B-mode imaging, in that the ultrasound system fires and receives a series of beams to scan a field of view. However, since F-mode imaging requires calculation of the velocity of targets, each line is fired and received several times. As with B-mode imaging, the data returned from each firing of each line is used to reconstruct an image on a console.

F-mode imaging is often used concurrently with B-mode imaging. For example, the gray scale image reconstructed from a B-mode scan can be superimposed with an F-mode image reconstructed from an F-mode scan of the same field of view or of a lesser included field of view. The F-mode information can be displayed using colours, with different colours indicating different positive or negative flow velocities or turbulence at the part of the B-mode image on which the pixel is superimposed. Because F-mode imaging is intended to provide only qualitative insight into target motion in the patient's body, the ultrasound system's processing of F-mode signals need not have high spatial or velocity resolution either in amplitude or in pixel resolution. However, since an important value of F-mode imaging is to detect flows relative to anatomical structures in the body, it is usually important that the P-mode image be properly registered with the B-mode image on-screen. Since this technique relies on the correlation of signal obtained from one pulse versus the subsequent pulse, and since microbubbles can be destroyed by the first pulse, an F-signal is generated that is not related to motion. This loss of correlation can be shown in a variety of display formats but is typically displayed in colour.

Additional techniques contemplated for use in the present invention are well known in the art, and are described, for example, in Gamsu et al., Diagnostic Imaging Review (W. B. Saunders Co 1998).

Ultrasonic energy may be applied to at least a portion of the subject to image the target tissue. A visible image of an internal region of the subject may then be obtained, such that the identification of the lymph nodes can be ascertained.

Generally, images may be acquired at different time-points, the variable temporal resolution ranging from milliseconds to several minutes or at a single time point. Further, ultrasound data sets may be acquired in 2 or 3 spatial dimensions (2D and 3D data). Preferably, ultrasound data sets are acquired in 3 spatial dimensions and the processing of according to the method of the invention is carried out with 3D data as lymph nodes are only of small dimensions.

After acquisition of ultrasound images of the lymph nodes, the images are processed in such a way that quantitative measure of the contrast enhancement pattern of said lymph nodes are generated.

As described before, images may be acquired at different time-points. The processing of said images according to the method of the invention may be based on an image acquired at one single time point (static analysis) or on a series of images acquired at different time points (dynamic analysis).

As described in the introduction of this application, it was disclosed in the International Application No. PCT/NO03/00328 that benign sentinel lymph nodes appear uniformly echogenic, while malignant sentinel lymph nodes demonstrate a heterogenic contrast enhancement pattern with both areas of increased echogenity and areas that do not enhance. Without being bound to this theory, we believe that areas in a lymph node that do not enhance after an ultrasound contrast agent has accumulated within said lymph node are areas representing malignant tissue. In other words, images of malignant lymph nodes will contain a higher fraction of dark areas than images of normal nodes. Hence one preferred way of processing the acquired images according to the method of the invention is to generate a grey level histogram. A grey level histogram is a function which shows for each discrete grey level (typically integer values in the range 0-255) the number of pixels in the image that has that grey level, corresponding to the logarithm of the local intensity of the reflected ultrasound signal. The centre of gravity of such a histogram is the mean grey level value of the region of interest, but is expected also to vary with instrument gain settings and the unknown attenuation of ultrasound in the tissues. This might be used as an indicator of malignancy; however, descriptive statistical indices such as standard deviation, skewness and kurtosis are independent of attenuation and gain and hence are preferred. These values are preferably compared with corresponding values for normal lymph nodes.

The spatial size of image details in normal contrast enhanced lymph nodes is dominated by normal ultrasound image “speckle” while image details in malignant nodes are dominated by variations due to larger dark regions representing malignant tissue. Thus a further preferred embodiment of the invention is to process the acquired ultrasound images in such a way, that quantitative measures of contrast enhancement pattern coarseness are generated. Several suitable methods can be used for images processing. In a preferred embodiment, the measure is based on borderline length measures. Only the region of the image that is identified as a lymph node is processed. The region is first segmented into areas that represent normal tissue (contrast enhanced) and potential malignant tissue (non-enhanced). The segmentation is preferably based on local image brightness, typically by comparing local brightness with a threshold which is derived from the actual span of brightness values in the whole lymph node. In a preferred embodiment, the threshold is the mean or the median of pixel brightness values. In a next step, the length of the borderline between the dark and the bright areas is measured, and is divided by the total area of the region. A coarse pattern of bright and dark regions associated with a malignant lymph node gives a short borderline, while a finely granulated pattern gives a longer borderline. Optionally, the image might be smoothed by a two-dimensional low-pass filter before the above-described processing is performed. Such a low-pass filter might also be non-parametric, e.g. a median filter.

In normal lymph nodes, contrast enhancement upon inflow and accumulation of the contrast agent follows a specific pattern (hereinafter called filling pattern). Early after administration, i.e. when the ultrasound contrast agent has just begun to enter the lymph node under examination, the ultrasound image shows a brighter “rim” around a central darker core. Somewhat later, i.e. after the contrast agent has accumulated within the lymph node, the node appears more uniformly bright. The filling pattern in malignant lymph nodes containing areas of metastatic tissue differs from the filling pattern in normal lymph nodes, and this difference can be quantified. Hence a further preferred embodiment of the method according to the invention is to process the acquired ultrasound images in such a way, that quantitative measures of the filling pattern are generated.

In one preferred embodiment, said processing is based on one image selected from a series of images acquired in the period beginning shortly after administration of the contrast agent and ending at complete accumulation of the contrast agent within the lymph node. Preferably, the selected image displays the contrast enhancement pattern at a time point after administration where the difference in said contrast enhancement pattern between a normal and a malignant node is a maximum difference. In a first step of said preferred embodiment, a template is generated based on the appearance of a typical normal lymph node at this time point after contrast agent administration. In a second step, the template is geometrically fitted to the selected ultrasound image of the lymph node under examination. Generally, geometrical fitting methods are known in the art and involve for instance translation, rotation, distortion and scaling of the standard. Typically, an affine transformation will be used. The geometrical fitting is optimised until the best fit is achieved. Automatic fitting procedures based on a general error minimisation algorithm are preferred. The measure of goodness of fit to be optimised might be based on the correlation between geometrically corresponding pixel brightness values from the template and the image. In a third step, a goodness of fit parameter is calculated, typically by taking the optimal value of the goodness of fit function of step 2. If necessary, the parameter is scaled to be dimensionless in order to not being dependent on the area of the image or the mean pixel brightness. Normal lymph nodes will have a close to optimal value (good fit) of the parameter while malignant lymph nodes will suboptimal values (poor fit).

In another preferred embodiment, the processing is based on a whole series of images or a part of said series of images acquired in the period beginning shortly after administration of the contrast agent and ending at complete accumulation of the contrast agent within the lymph node. As described above, the filling pattern of a normal lymph node is as such that the rim or periphery of the lymph node enhances first and the enhancement gradually extends towards the centre of the lymph node. Hence the filling of a normal lymph node proceeds in a circular symmetrical fashion while the filling of a malignant lymph node proceeds in a more unsymmetrical or disorganised fashion. Several methods may be applied to identify deviations from a standard filling pattern, i.e. the filling pattern of a normal lymph node. In a preferred embodiment, the temporal and/or spatial deviations of contrast appearance between peripheral and central regions of the lymph node is estimated, preferably by fitting a model to the lymph node with concentric regions from the periphery to the centre, calculating the time of contrast appearance for each region (e.g. time to half of maximum intensity) and subsequently calculating the difference in appearance times. In another preferred embodiment, the temporal development of the goodness of fit parameters calculated as described in the last preceding paragraph is used to further exploit the dynamic information available. Deviations of the parameters with respect to the expected values from normal lymph nodes are used for classifying the node into malignant or non-malignant.

As mentioned before, ultrasound images of normal lymph nodes show almost homogeneous contrast enhancement, however, still containing normal ultrasound speckle. Such images also show an irregular radially oriented streaky pattern. Images of malignant nodes however show a more random pattern of dark and bright regions. These differences in contrast enhancement pattern or, more precisely, texture of the lymph node can be quantified. Hence a further preferred embodiment of the method according to the invention is to process the acquired ultrasound images in such a way, that quantitative measures of the texture of the lymph node are generated. In a first step of a preferred embodiment, a region of interest (ROI) is selected on the image displaying the lymph node under examination said ROI encompasses the lymph node. The ROI may be selected by an operator or by using an automatic technique. Preferably, speckle is removed form the ROI, e.g. by spatial low-pass filtering. However, depending on the setting of the ultrasound scanner (e.g. frequency, imaging bandwidth), this may not be necessary and can be omitted. The ROI is analysed for orientation of pattern.

Several mathematical methods can be used to carry out the analysis and these methods can be applied to both 2D and 3D images. A preferred method for carrying out the analysis is a Fourier based method where the spatial Fourier transform is calculated in the ROI. Normal and malignant lymph nodes are discriminated by their spatial power spectrum. Preferably, they are discriminated by their spectral bandwidth or extension in k-space. While a normal lymph node shows a background of high frequency spatial components (representing the random, homogeneous speckle), and discrete spectral peaks representing the repeated oriented lines described above, a malignant node will show a preponderance of low frequency components representing the larger regions of malignant tissue.

In another preferred embodiment, spatial autocorrelation is used for carrying out the analysis for orientation of pattern. The autocorrelation function is calculated in 2D or 3D space.

In a further preferred embodiment, quantitative measure of the texture is combined with quantitative measure of pattern coarseness. Hence a further preferred embodiment of the method according to the invention is to process the acquired ultrasound images in such a way that quantitative measures of pattern coarseness and the texture of the lymph node are generated.

In another preferred embodiment, neural networks are applied to discriminate between normal and malignant lymph nodes in the method according to the invention. Neural networks are preferably applied to images with segmented areas of enhancement and non-enhancement in the lymph node images as described above.

Another aspect of the invention is a method of processing ultrasound images of lymph nodes from a subject preadministered with an ultrasound contrast agent wherein quantitative measures of the contrast enhancement pattern of said lymph nodes are generated and these measures are used to characterise the lymph nodes as being normal or malignant lymph nodes.

Yet another aspect of the invention is a method of characterising lymph nodes as being normal or malignant lymph nodes by administering an ultrasound agent to a subject, acquiring ultrasound images of the lymph nodes of said subject, processing said images in such a way that quantitative measures of the contrast enhancement pattern of said lymph nodes are obtained.

The characterisation is based on either the quantitative measures obtained alone or by comparing said quantitative measures with the corresponding quantitative measure of a normal lymph node.

The invention will now be described in greater detail by reference to the following non-limiting examples.

### EXAMPLES

### Example 1

### In Vivo Sentinel Lymph Node Detection and Characterisation

Anaesthetised Sinclair pigs with melanoma tumours were used to investigate sentinel lymph node detection and characterisation using Sonazoid™.

1 ml Sonazoid™ was administered intradermally around each primary tumour in a Sinclair pig with 3 melanoma tumours. After 5 minutes of gentle massage of the injection site, ultrasound scanning was performed with a Siemens Elegera scanner. The sentinel lymph node showed strong contrast enhancement on high frequency pulse inversion digital grey scale imaging. One of the lymph nodes showed a homogenous contrast enhancement pattern, while the other two showed a spotty, heterogeneous enhancement pattern. Upon visual assessment of the ultrasound images, the first node was characterised as normal, while the two latter nodes were characterised as malignant. Microscopic histological examination of the lymph nodes confirmed absence of tumour in the first node and the presence of tumour in the two latter nodes.

The image of the normal lymph node is slightly smoothed by spatial low-pass filtering, removing the random speckle of the image while preserving the anatomical information. The portion of the image that contains the lymph node is used as a template for analysing the two nodes which have been characterised as malignant.

### Example 2

### Analysis of Lymph Node Image by Template Correlation

An iterative algorithm, such as the downhill simplex method of Nelder and Mead is used to align the template of Example 1 with the images of the malignant nodes of Example 1. Motion, size scaling, skewing and rotation of the template with respect to the malignant lymph node images is performed by an affine transformation with six degrees of freedom, and the parameters that describe the transformation are automatically adjusted for optimum goodness of fit by the algorithm. The goodness of fit parameter is calculated as the correlation coefficient between geometrically corresponding pixel grey scale values of the malignant lymph node images and the template image. The maximum value of the goodness of fit obtained by the algorithm is used as a measure of how well the lymph nodes resemble the template. A low value, typically below 0.8, indicates metastatic growth in the node (i.e. a malignant lymph node).

### Example 3

### Analysis Based on Borderline Length

An image of a lymph node is acquired as described in Example 1 about 10-20 minutes after injection. The image is smoothed as described in Example 1, and the region of interest (ROI) in the image that contains the lymph mode is defined by the user. The median pixel grey level value in this region is calculated, and the pixels in the region are segmented as dark or bright dependent on whether they are darker or brighter than the median value. A measure of the length of the borderline between bright and dark regions is calculated by traversing the rows of image pixels that are inside the ROI, and counting the total number of transitions between pixels that are classified as dark and bright. The borderline length is then estimated to be:

$${L}_{b} = \frac{π {D}_{p} n}{2}$$

Where Dₚ is the distance between pixel rows, and n is the number of transitions. In order to create a measure that is independent on the size of the lymph node, the length estimate is divided by the area of the region. A result that is lower than reference values obtained from normal lymph nodes indicates the presence of metastatic tissue.

### Example 4

### Analysis Based on the Autocorrelation Function

An image of a lymph node is acquired as described in Example 3 and smoothed as described in Example 1. The normalised 2D autocorrelation function of the ROI image is calculated either by Fourier transform techniques (inverse transform of the 2D power spectrum), or by discrete calculation techniques. The size of the autocorrelation peak is estimated as the number of pixel in the autocorrelation matrix with values above 0.5. Pixels that represent local peaks not connected to the primary peak of the autocorrelation function are disregarded A number that is higher than corresponding to an image area of about 4 mm² indicates a coarse texture caused by regions of metastatic tissue in the node.

What is claimed is:

1. A method of processing ultrasound images of lymph nodes from a subject preadministered with an ultrasound contrast agent wherein quantitative measures of the contrast enhancement pattern of said lymph nodes are generated.

2. A method according to claim 1 wherein the ultrasound contrast agent used for preadministration is a particular ultrasound contrast agent.

3. A method according to claim 2 wherein said particular ultrasound contrast agent comprises a matrix material and a gas, a gas precursor or mixtures thereof.

4. A method according to claim 3 wherein said particular ultrasound agent has a mean particle size of about 0.25-15 μm in diameter and a pressure stability of at least 50% at a pressure of 120 mm Hg.

5. A method according to claim 1 wherein said ultrasound images were acquired at one single time point or at different time points.

6. A method according to claim 1 wherein said ultrasound images were acquired in 2 or 3 spatial dimensions.

7. A method according to claim 1 wherein the ultrasound images are processed in such a way that grey level histograms are generated from the contrast enhancement pattern of said images.

8. A method according to claim 7 wherein descriptive statistical indices are calculated from the grey level histograms, preferably standard deviation, skewness or kurtosis.

9. A method according to claim 1 wherein the ultrasound images are processed in such a way that quantitative measures of contrast enhancement pattern coarseness are generated.

10. A method according to claim 9 wherein said processing is based on borderline length measures.

11. A method according to claim 9 comprising
a) identification of the region of said images which show a lymph node
b) segmentation of said region into contrast enhanced and non-enhanced areas, and
c) measuring of the length of the borderline between the enhanced and non-enhanced areas and dividing the obtained measure by the total area of said region.

12. A method according to claim 1 wherein the ultrasound images are processed in such a way that quantitative measures of the contrast enhancement of the filling pattern are generated.

13. A method according to claim 12 wherein said processing is based on one single image.

14. A method according to claim 13 comprising
a) generating a template based on an image of a normal lymph node
b) geometrically fitting said template to said image of the lymph node from the subject, and
c) calculating the optimal goodness of fit parameter.

15. A method according to claim 12 wherein said processing is based on a series of images.

16. A method according to claim 15 comprising the estimation of temporal and/or spatial deviations of contrast enhancement appearance between peripheral and central regions of said lymph nodes.

17. A method according to claim 15 comprising
a) fitting a model to said lymph nodes with concentric regions from the periphery to the centre
b) calculating the time of contrast enhancement appearance for each of the concentric regions, and
c) calculating the difference in contrast enhancement appearance time.

18. A method according to claim 1 wherein the ultrasound images are processed in such a way that quantitative measures of the texture of the lymph nodes are generated.

19. A method according to claim 18 comprising
a) selecting a region of interest on said images displaying the lymph nodes from the subject, and
b) analysing the selected region of interest for orientation and spatial frequency of pattern

20. A method according to claim 19 wherein the spatial Fourier transform is calculated in step b).

21. A method according to claim 19 wherein the autocorrelation function is calculated in step b).

22. A method according to claim 1 wherein the ultrasound images are processed in such a way that quantitative measures of the contrast enhancement pattern coarseness and the texture of the lymph nodes are generated.

23. A method according to claim 1 wherein the quantitative measures are used to characterise the lymph nodes as being normal or malignant lymph nodes.

24. A method according to claim 14 wherein the goodness of fit parameter calculated in step c) is compared with a threshold value in order to characterise the lymph node as a normal or malignant lymph node.

25. A method according to claim 17 wherein the results of step c) are used to characterise the lymph node as a normal or malignant lymph node.

26. A method of characterising lymph nodes as being normal or malignant lymph nodes by administering an ultrasound agent to a subject, acquiring ultrasound images of the lymph nodes of said subject and processing said images in such a way that quantitative measures of the contrast enhancement pattern of said lymph nodes are obtained.
